(19) United States
(12) Patent Application Publication
(10) Pub. No.: US 2007/0270661 A1
(43) Pub. Date: Nov. 22, 2007

(54) Method and apparatus for monitoring blood flow to the hip joint

(76) Inventors: Marc Philippon, Edwards, CO (US); Allston Stubbs, Brookline, MA (US)

Correspondence Address: HELLER EHRMAN LLP, 275 MIDDLEFIELD ROAD, MENLO PARK, CA, 94025-3506, US

(21) Appl. No.: 11/391,844

(22) Filed: Mar. 28, 2006

(51) Int. Cl. A61B 5/00

(52) U.S. Cl. 600/300

(57) ABSTRACT

Devices, systems and methods for performing arthroscopic evaluations and procedures in and near the hip joint are provided. An arthroscopic probe having a Doppler probe disposed on a movable tip portion is useful for determining blood flow and for identifying vascular structures. An arthroscopic probe having a Doppler probe and a radiofrequency (RF) source disposed on a movable tip portion provides further useful features including the ability to perform cauterization, tissue ablation, and other RF procedures. The devices, systems, and methods are effective to assist an operating surgeon in the assessment of femoral head blood supply before, during, and after procedures involving the hip joint and optionally to provide for RF procedures in concert with such assessments of blood flow. The devices, systems, and methods of the present invention aid in arthroscopic procedures, and are useful in reducing the risk of iatrogenic hip joint avascular necrosis (AVN).

### FIELD OF THE INVENTION

This invention relates to devices, systems and methods for arthroscopic examination and treatment, particularly in and near the hip joint. In particular, the invention relates to arthroscopic devices, systems and methods for monitoring blood flow to the hip joint and to the region near to the hip joint, and for ablative and other arthroscopic medical treatments in and near the hip joint.

### BACKGROUND TO THE INVENTION

The hip is vital to human locomotion, and hip injuries and diseases can significantly impact the ability of a patient to carry out day-to-day tasks as well as impair the performance of athletes and active amateur sports enthusiasts. Hip conditions impairing movement or causing pain with normal activity may result from trauma, age, or disease conditions.

Hip surgery is indicated where there is injury or change in the hip joint requiring removal or reshaping of bone or cartilage or of material present in the joint. Arthroscopic surgery causes the least amount of ancillary trauma and allows for more rapid recovery than do other forms of hip surgery. Arthroscopic treatment of the hip is discussed, for example, in Kelly et al., “Hip Arthroscopy: Current Indications, Treatment Options, and Management Issues,” American Journal of Sports Medicine 31(6):1020-1037 (2003).

Avascular necrosis (AVN) is a common disease of the hip joint. Prevention of AVN is important as treatment of hip avascular necrosis is difficult. AVN of the hip, and more specifically the femoral head, is secondary to unknown and known causes. The known causes comprise alcohol use, steroid use, hemoglobinapathies, metabolic syndromes, accidental trauma, and iatrogenic injury. Current operative techniques about the hip may place the blood supply at risk and be a source of iatrogenic injury leading to AVN. Specifically, surgical efforts to restore joint mechanics and congruency may compromise blood flow and lead to osteonecrosis.

The anatomy, physiology, and possible sources of injury related to AVN of the hip are discussed, for example, in Crock H V, “A revision of the anatomy of the arteries supplying the upper end of the human femur,” J Anat 99:77-88, 1965; Beck M et al., “Increased intraarticular pressure reduces blood flow to the femoral head,” Clin Orthop Relat Res 424:149-52, 2004; Gautier E et al., “Anatomy of the medial femoral circumflex artery and its surgical implications,” JBJS 82-B:679-683, 2000; Layernia C J et al., “Osteonecrosis of the femoral head,” JAAOS 7: 250-261, 1999; and Steinberg M E, Hayken G D, Steinberg D R, “Classification and staging of osteonecrosis,” In Urbaniak J R, Jones J P (eds). Osteonecrosis: Etiology, Diagnosis, and Treatment, published by Rosemont, American Academy of Orthopaedic Surgeons 277-284, 1997.

Although arthroscopic examination, evaluation, and treatment of the hip and hip region are common procedures, such procedures present risk of damage to arteries and nerves which are present in the region. Such structures are often not readily identified or properly distinguished during arthroscopic procedures, unnecessarily prolonging such procedures or even allowing mistakes and inadvertent injury to the patients undergoing such procedures.

The most common arthroscopic approach to the hip is from the anterior aspect of the thigh. The two arthroscopic portals from this approach are the anterior and anterior-lateral. Occasionally, a third portal is utilized, the accessory lateral portal. A hip arthroscopy involves procedures within two compartments, the central (intraarticular) and the peripheral. From the central compartment, the surgeon can address pathology of the labral and articular cartilage, the synovium (joint lining), and acetabular rim. From the peripheral compartment, the surgeon can address pathology of the femoral head and neck junction. It is along this junction that the blood supply reaches the femoral head and is at risk.

Accidental contact with, occlusion of, or injury to, vascular structures in the hip region during medical procedures including arthroscopic procedures may lead to AVN or other conditions. For example, iatrogenic injury is thought to arise from compromise of the medial femoral circumflex artery which supplies a majority of blood flow to the femoral head and may be a cause of AVN. The actual number of cases involving surgeon-induced hip AVN is unknown; however, the risk of surgical injury is increasing with the increase in arthroscopic hip procedures which seek to reshape the femoral head.

There is therefore a need for improved devices and methods for and for improved arthroscopic treatments which lower the risk of iatrogenic injury and provide improved means for performing arthroscopic hip treatments and procedures.

### SUMMARY OF THE INVENTION

This invention relates to devices, systems and methods for medical and veterinary arthroscopic examination and treatment, and are particularly useful for medical procedures in and near a hip joint of a patient. The patient may be a mammal, and is preferably a human patient.

The devices may be disposable, single use arthroscopic devices. The devices, systems and methods disclosed herein are useful for the measurement of blood flow in and near a hip joint, and may also aid in radiofrequency treatments, including cauterization and ablation of tissue, in and near a hip joint. Embodiments of the devices, systems and methods are effective to assist an operating surgeon in the assessment of femoral head blood supply before, during, and after procedures involving a hip joint of a patient in need of such treatment or in the examination of a hip joint suspected to be in need of such treatment. Such monitoring is effective to reduce accidental damage to blood vessels in and near a hip joint during medical procedures such as arthroscopic examination and arthroscopic treatment, so as to reduce the risk of causing or exacerbating hip joint avascular necrosis (AVN).

In an embodiment, the present invention provides an arthroscopic probe for a hip procedure having a shaft with a longitudinal axis defining a proximal direction, a distal direction and radial directions perpendicular to said longitudinal axis, a handle portion with a proximal end and a control element disposed at or near the proximal end of said shaft, said shaft having a tip portion with a distal tip and a Doppler transducer effective for detecting blood flow in a blood vessel, the distance between said distal tip and said handle proximal end defining a probe length, said control element being operably connected with said tip portion, the tip portion being movable with respect to said longitudinal axis at a deflection angle to the shaft, said deflection angle being controllable by the control element. The tip portion may be deflected from an orientation that is substantially parallel to the longitudinal axis of the shaft by a deflection angle of between about 0° and about 160°, or of between about 0° and about 120°, or of between about 0° and about 90°, or of between about 0° and about 45°. In some embodiments, the deflection of the tip is in a downward direction with respect to the longitudinal axis of the shaft, where a downward direction is defined as being in a direction similar to the direction of the handle with respect to the shaft axis.

The movable tip portion may include a flexible region proximal to the distal tip of the probe. Movement of the movable tip may be effected by movement or flexion of the flexible region. The movable tip may have a length of between about 2 mm and about 75 mm, or between about 5 mm and about 50 mm, or between about 10 mm and about 30 mm, or between about 15 mm and about 25 mm. The tip portion typically has a width of between about 1 mm and about 20 mm. The tip portion may have a substantially circular cross-sectional shape, a substantially elliptical cross-sectional shape, a substantially rectangular cross-sectional shape, and irregular cross-sectional sh may have a tip diameter of between about 2 mm and about 20 mm, or between about 3 mm and about 10 mm, or between about 4 mm and about 8 mm. A tip portion with a substantially elliptical cross-sectional shape has a larger elliptical diameter and a smaller elliptical diameter. A tip portion having a substantially elliptical cross-sectional shape may have a larger elliptical diameter of between about 3 mm and about 10 mm and a smaller elliptical diameter of between about 1 mm and about 8 mm, or a larger elliptical diameter of between about 4 mm and about 8 mm and a smaller elliptical diameter of between about 2 mm and about 6 mm.

The distance between the distal tip and the proximal extent of the handle of an arthroscopic hip probe having features of the invention defines a probe length. A probe length may be between about 100 mm and about 500 mm, or between about 200 mm and about 400 mm, or between about 250 mm and about 350 mm.

The Doppler transducer of an arthroscopic hip probe having features of the invention may be an ultrasound Doppler transducer, an electromagnetic radiation Doppler transducer, or other Doppler transducer suitable for Doppler measurements of blood flow. For example, an electromagnetic Doppler transducer may be an infrared Doppler transducer. Devices and systems having features of the invention may comprise a source of ultrasound, electromagnetic (e.g., infrared), or other radiation or energy suitable for Doppler measurements of blood flow. In embodiments of the devices and systems disclosed herein, a power supply or source of energy suitable for Doppler measurements of blood flow, is operably connected to a Doppler transducer of the device, and may be incorporated in or provided with the device or system.

An arthroscopic hip probe having features of the invention may include a source of radiofrequency (RF) energy, such as an RF electrode. An arthroscopic hip probe having features of the invention having an RF electrode may have a monopolar RF electrode or a bipolar RF electrode, or both. Where an arthroscopic hip probe having features of the invention has a monopolar RF electrode, a ground electrode, such as a ground pad, is also operably connected to the device or system, and may also be provided with the device or system. In embodiments of the devices and systems disclosed herein, an RF power supply effective to provide RF energy to an RF electrode is operably connected to an RF electrode of the device, and may be incorporated in or provided with the device or system.

Thus, an arthroscopic probe for a hip procedure having features of the invention has a Doppler means for detecting blood flow in a blood vessel, and has a control means effective to move the movable tip of the probe. An arthroscopic probe for a hip procedure having features of the invention may also have a radiofrequency means for providing radio frequency energy for use during said hip procedure.

Also provided is a system for a hip procedure, comprising an arthroscopic probe as discussed above, and including another element selected from one or more of a power supply for a Doppler probe, an audio monitor output for a Doppler probe, a visual monitor output for a Doppler probe, a processing device for processing signals from a Doppler probe, and an access cannula. The probe may be operably connected to an amplifier, audio monitor, or computational device via a wire, or via a wireless connection. A system for a hip procedure may include a power supply for a radiofrequency probe, a grounding pad for a monopolar radiofrequency probe, and a control for a RF probe. An RF probe may be used to heat, to coagulate, to weld together, or to ablate tissues. Control elements for systems for a hip procedure may include hand or foot controls (e.g., hand operated switches, or foot pedals, to control tip configuration and/or to control RF electrode functions).

Also provided are methods for performing hip procedures at or near a hip joint. In embodiments, a method of performing an arthroscopic procedure at or near to a hip joint of a human patient, comprises a) inserting an arthroscopic hip probe into a patient near a hip joint of the patient, said arthroscopic hip probe having a shaft and a Doppler probe disposed on a movable tip of said shaft; b) moving said movable tip with respect to the axis of said shaft of said hip probe effective to place said Doppler probe adjacent a blood vessel of said patient near a hip joint of said patient; and c) monitoring blood flow in said blood vessel. In embodiments of the methods, the arthroscopic hip probe is an arthroscopic hip probe having features of the invention, such as, for example, an arthroscopic hip probe having a movable tip and a Doppler transducer, and may have an electrode for providing RF energy. The methods may further comprise d) applying RF energy to tissue near a hip joint of said patient. Such tissue to which RF may be applied may be one or more of connective tissue, bone, muscle tissue, and vascular tissue. The vascular tissue may be, for example, either or both of arterial tissue or venous tissue.

The devices, systems and methods for arthroscopic examination and treatment disclosed herein provide improved tools for the operator and increased safety for the patient.

### BRIEF DESCRIPTION OF THE FIGURES

FIG. 1 shows a schematic view of a Doppler probe system having features of the invention including a Doppler probe configured for use in arthroscopic evaluations and treatments of the hip and having features of the invention including a movable tip portion.

FIG. 2 shows a schematic view of a Doppler probe plus radiofrequency (Doppler-RF) system having features of the invention including a Doppler-RF probe configured for use in arthroscopic evaluations and treatments of the hip and having features of the invention including a movable tip portion.

FIG. 3A shows a schematic view of a hip probe configured for use in arthroscopic evaluations and treatments of the hip and having features of the invention. A hip probe of FIG. 3A may be either a Doppler hip probe or a Doppler-RF hip probe, and may have tip portions having features of any of FIGS. 3B-3F.

FIG. 3A shows a schematic view of a hip probe configured for use in arthroscopic evaluations and treatments of the hip and having features of the invention.

FIG. 3B is a schematic illustrations of a tip portion of a hip probe of FIG. 3A in which the tip portion is displaced downwardly by a deflection angle of about 45°.

FIG. 3C is a schematic illustrations of a tip portion of a hip probe of FIG. 3A in which the tip portion is displaced downwardly by a deflection angle of about 90°.

FIG. 3D is a schematic illustrations of a tip portion of a hip probe of FIG. 3A in which the tip portion is displaced downwardly by a deflection angle of about 120°.

FIG. 3E is a schematic illustrations of a tip portion of a hip probe of FIG. 3A in which the tip portion is displaced downwardly by a deflection angle of about 150°.

FIG. 3F is a schematic illustrations of a tip portion of a hip probe of FIG. 3A in which the tip portion is displaced downwardly by a deflection angle of about 170°.

FIG. 4A is a schematic view of a tip portion of a movable tip having circumferential gaps in portions of the shaft wall.

FIG. 4B is a cut-away view taken along line 4B-4B showing the tip portion of the movable tip of FIG. 4A in longitudinal section.

FIG. 4C is a cut-away view taken along line 4C-4C showing the tip portion of the movable tip of FIG. 4A in cross-section, showing the lack of material within a gap.

FIG. 4D is a cut-away view taken along line 4D-4D showing the tip portion of the movable tip of FIG. 4A in cross-section, showing the circumferential continuity of the material in a portion between gaps.

FIG. 4E is a schematic view of a tip portion of a movable tip having angled gaps in portions of the shaft wall.

FIG. 4F is a schematic view of a tip portion of a movable tip having gaps in portions of the shaft wall in which the gaps have wider portions and thinner portions.

FIG. 4G is a schematic view of a tip portion of a movable tip having pleats in portions of the shaft wall.

FIG. 4H is a schematic view of a tip portion of a movable tip having mesh in a portion of the shaft wall.

FIG. 4I is a schematic view of a tip portion of a movable tip in which a portion of the shaft wall includes a flexible material.

FIG. 5A shows a partially cut-away view of a hip probe configured for use in arthroscopic evaluations and treatments of the hip and having features of the invention, in which the movable tip is directed to deflect in a downward direction by a mechanical mechanism controlled by motion of a handle portion. A Doppler hip probe of FIG. 5A, 5B, 5C or 5D may be either a Doppler hip probe or a Doppler-RF hip probe.

FIG. 5B shows a partially cut-away view of a hip probe configured for use in arthroscopic evaluations and treatments of the hip and having features of the invention in which the movable tip is directed to deflect in a downward direction by a pneumatic mechanism controlled by motion of a handle portion.

FIG. 5C shows a partially cut-away view of a hip probe configured for use in arthroscopic evaluations and treatments of the hip and having features of the invention in which the movable tip is directed to deflect in a downward direction by a mechanical mechanism controlled by motion of a syringe-type handle portion.

FIG. 5D shows a partially cut-away view of a hip probe configured for use in arthroscopic evaluations and treatments of the hip and having features of the invention, in which the movable tip is directed to deflect in a downward direction by an electrical mechanism controlled by motion of a handle portion.

FIG. 6A shows a schematic view of a tip portion of a Doppler probe having features of the invention viewed from distal point looking in a proximal (towards the handle) direction showing the distal end portion of the Doppler probe.

FIG. 6B shows a schematic view of a tip portion of a Doppler-RF probe (monopolar RF) having features of the invention viewed from distal point looking in a proximal (towards the handle) direction showing the distal end portion of the Doppler transducer disposed in a central position of the probe tip and showing the distal end portion of the monopolar RF probe disposed around the Doppler transducer.

FIG. 6C shows a schematic view of a tip portion of a Doppler-RF probe (monopolar RF) having features of the invention viewed from distal point looking in a proximal (towards the handle) direction showing the distal end portion of the Doppler transducer disposed in a central position of the probe tip and showing the distal end portion of the bipolar RF probe disposed around the Doppler transducer.

FIG. 6D shows a schematic view of a tip portion of a Doppler-RF probe (bipolar RF) having features of the invention viewed from distal point looking in a proximal (towards the handle) direction showing the distal end portion of the Doppler transducer disposed in a central position of the probe tip and showing the distal end portion of the bipolar RF probe disposed around the Doppler transducer.

### DETAILED DESCRIPTION OF THE INVENTION

The scope of hip arthroscopy is increasing to include procedures which modify both the bone and soft tissue structures of the hip. In modifying the bone structure of the femoral head and neck junction, one risks compromising the blood supply to the femoral head which might lead to AVN. Currently, there is no practical method or device to assess the blood flow to the femoral head during an arthroscopic procedure. To avoid injury to the medial femoral circumflex artery, the surgeon must rely on variable anatomic relationships along the femoral head and neck junction. To both the inexperienced and experienced surgeon, a reliable method to assess blood flow to the femoral head before, during, and after an arthroscopic procedure would be important and useful.

Apparatus

The present invention includes an arthroscopic probe that is capable of extended passage through the deeper soft tissues of the hip via a standard arthroscopic portal. The distal tip may have a curved and flexible capability to adapt to the curvilinear spaces of the hip's peripheral compartment. In embodiments, the distal tip is flexibly attached to a shaft and is movable with respect to the shaft, so that the tip may be placed as desired at any one of several angles with respect to the shaft axis. In embodiments, an arthroscopic probe having features of the invention is a disposable, single use device. The most distal end of the probe contains a Doppler transducer able to detect motion, such as blood flow (also termed “vascular flow”) and able to detect changes in vascular flow. The probe may connect to an operating room monitor via its cable or via a wireless connection. The monitor may emit a sound and/or a visual signal related to the Doppler measurements, effective to indicate the presence and optionally magnitude or direction of motion, such as vascular flow, detected with the Doppler transducer.

Method

The medial femoral circumflex artery (MFCA) is the primary blood supply to the femoral head. The MCFA and its terminal subsynovial branches can be assessed via the peripheral compartment of the hip through the standard anterior portals. Like most arteries, this assessment is best conducted using an atraumatic, noninvasive method. The present invention utilizes the Doppler effect (e.g., utilizes changes in sound or light waves) to detect vascular flow. Detection of vascular flow near the probe tip aids guidance of the probe and aids in the determination of the probe's location within a patient. Detection of vascular flow near the probe tip also aids in identification of blood vessels, and differentiation between blood vessels, nerves, and other internal structures, and so aids in medical procedures using a probe having features of the invention.

In use, a Doppler probe with flexible capabilities (e.g., a movable tip portion) is placed along the posterior-lateral femoral neck in the region of the MCFA subsynovial branches. The Doppler probe is disposed on or near the tip of the probe shaft, the tip being moveable with respect to the shaft. A major portion of the probe shaft is typically straight, although in embodiments at least a portion of the shaft may be curved. A triphasic Doppler flow signal indicates positive blood flow. For example, a triphasic Doppler flow signal in the region of the MCFA subsynovial branches indicates positive blood flow along the femoral neck and to the femoral head. A blood-flow assessment can be made at any point in the procedure.

Application

The systems, apparatus and methods of the present invention are particularly useful for hip procedures in which the operating surgeon considers the MCFA and its terminal branches at risk and thus the need for active arterial monitoring to reduce the future possibility of AVN. Such procedures include but are not limited to hip arthroscopy, osteoplasty of the femoral head neck junction, fracture-dislocation reduction of the hip, and slipped capital femoral epiphyseal reductions. It will be understood that the systems, apparatus and methods disclosed herein may also find use in other medical procedures and therapeutic applications as well.

Definitions

Where the singular is used, it is to be understood that plural is also included, so that, for example, the terms “a probe” and “ari electrode” include and refer equally to multiple probes and electrodes as well as to a singular probe and a singular electrode.

As used herein, the terms “movable” and “flexible” refer to the ability of the object modified by such terms to alter or have altered, its position, such as its relative position with respect to another object, or to alter, or have altered, its shape.

As used herein, the term “connective tissue” refers to ligaments (which connect bone to bone) and tendons (which connect muscle to bone). Cartilage and cartilaginous structures, such as cartilage covering femur and pelvic bone in the hip joint, are included in the term “connective tissue.”

As used herein, the terms “bone” and “bone tissue” refer to the bones of a mammalian patient.

As used herein, the terms “muscle” and “muscle tissue” refer to skeletal and smooth muscle of a mammalian patient.

As used herein, the term “vascular tissue” refers to blood vessels and includes arterial and venous tissues and capillaries. Arterial vessels carry oxygenated blood from the heart and lungs to tissues, while venous vessels carry oxygen-depleted blood from the tissues to the heart and lungs. Capillaries are small vessels connecting the arterial with the venous system, and are the locus where oxygen transfer from blood to tissue typically occurs.

As used herein, the term “Doppler” refers to measurements utilizing the properties, especially the frequency, of reflected wave energy to determine the velocity of target objects. Particularly preferred target objects are moving blood cells, and a particularly preferred use of Doppler measurements is to detect the presence of blood vessels by detecting movement of blood cells therein using Doppler measurements. A further use is to differentiate between arterial vessels and venous vessels using Doppler measurements.

As used herein, an “emitter” is a source of wave energy; a “receiver” is a device or element that receives or detects wave energy; and a “transducer” is a device or element that transforms wave energy from one form to another, typically from one form to an electronic form that may be transmitted to another location, analyzed, stored (either digitally or in analog form), detected, broadcast (e.g., as a sound signal able to be heard by a human operator), or in other ways utilized.

As used herein, the term “wave energy” refers to any energy having periodic properties such as a frequency. Wave energy may be sound energy, which may be termed sonic, acoustic, ultrasound, or other energy, and may be audible to a human or may be inaudible to a human. Wave energy may be electromagnetic energy, such as electromagnetic radiation of the infrared, visible, ultraviolet, microwave, radiofrequency, or other spectral ranges.

As used herein, the term “radiofrequency energy” or equivalently “RF energy”, refers to electromagnetic energy having frequency and wavelength characteristics of radio energy. RF energy is useful in a medical or surgical setting, when applied to tissues, to coagulate or to ablate the tissues, and may be used to shape or repair tissues, to cauterize tissues, to weld tissues together, or to cut or dissect tissues.

Application of RF energy requires two electrical contacts in order to complete an electrical circuit in which electromagnetic energy flows through pr across the target tissue. A monopolar RF probe typically passes electromagnetic energy between a small, typically elongated conductive electrode applied to the target tissue and a ground pad having much greater surface area in contact with the patient's tissues (typically a patient's skin). A bipolar RF probe typically passes electromagnetic energy between a distal conductive electrode applied to the target tissue and a nearby electrode typically disposed around and somewhat proximal to the distal tip electrode.

Devices, systems and methods disclosed herein provide improvements in tools and methods useful for arthroscopic examination and treatment, particularly in and near a hip joint. The devices are suitable for arthroscopic procedures, are preferably disposable, and provide a unique combination of tip mobility and Doppler capability. The devices typically include an elongated shaft portion having a distal portion with a tip. A tip portion of the devices having features of the invention is configured to be movable with respect to other portions of the shaft, under the direction of the operator of the device. Operative elements may be disposed on a distal portion of the devices, and in embodiments may be disposed on a distal tip of the devices. In embodiments, the devices further include radiofrequency (RF) functionality. A Doppler transducer is disposed on a movable portion of the device at or near the distal tip of the device; an RF electrode or an RF electrode pair may also be disposed on a movable portion of the device at or near the distal tip of the device.

A shaft portion of a device having features of the invention may be straight, or may be curved, and may include both straight and curved portions. In embodiments, a shaft portion is straight and is flexible. In embodiments, a shaft portion may be configured to be able to be bent or curved by the hands of an operator, and to retain such bend or curve during use.

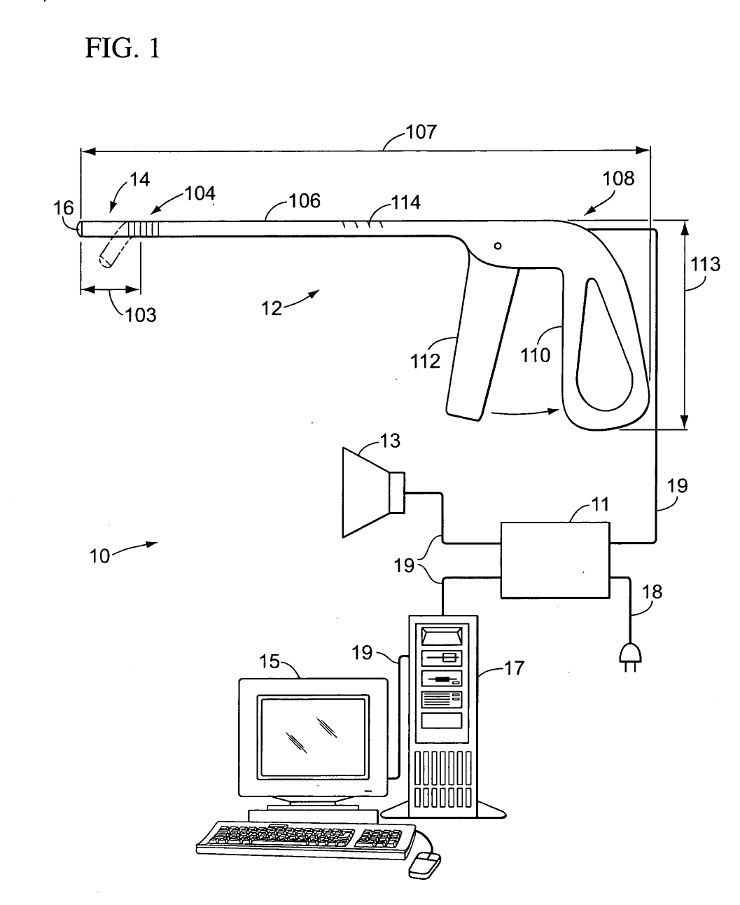

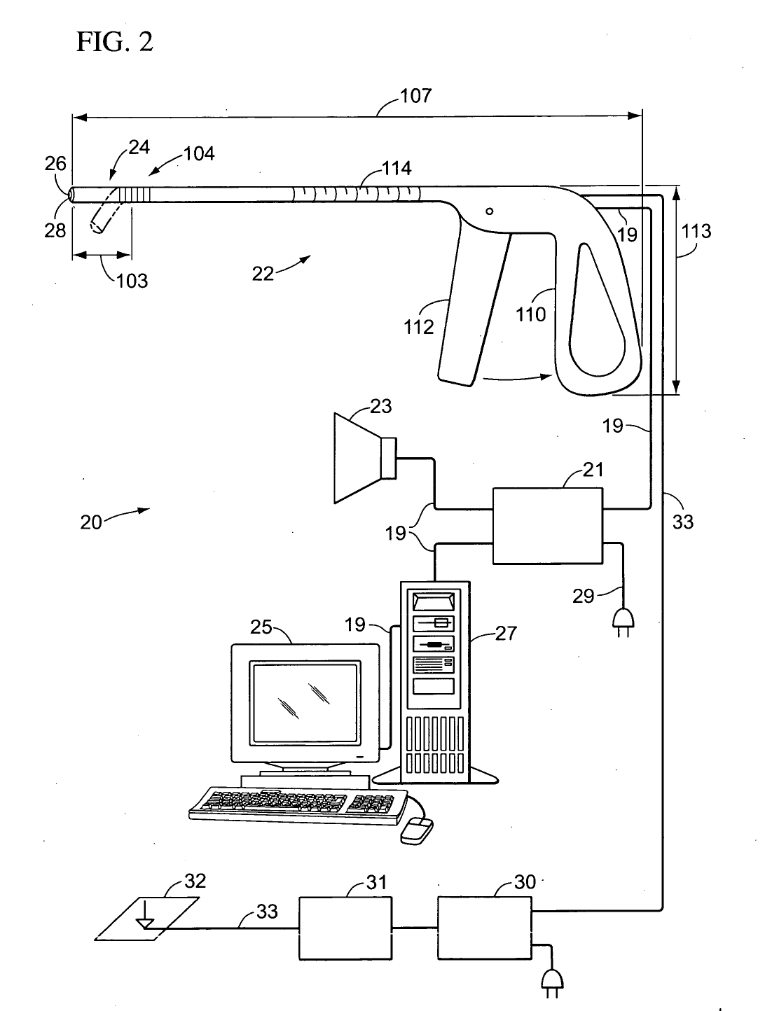

A Doppler system 10 including an arthroscopic probe 12 having features of the invention is illustrated in FIG. 1. A Doppler probe system 10 as illustrated in FIG. 1 includes a Doppler probe 12 having features of the invention configured for use in arthroscopic evaluations and treatments of the hip, the Doppler probe 12 having a movable tip portion 14, and a Doppler transducer 16. A Doppler-RF probe system 20 as illustrated in FIG. 2 includes a Doppler-RF probe 22 having a movable tip portion 24 with a Doppler transducer 26 and at least one radiofrequency (RF) electrode 28 configured for use in arthroscopic evaluations and treatments of the hip to provide a Doppler-RF system 20 having features of the invention.

Systems having features of the invention, such as a system 10 as illustrated in FIG. 1, may include Doppler power source 11, audio monitor 13, video monitor 15 (which may be a computer monitor operably connected to a standard computer), analysis module 17 (which may be, or include, a standard computer and computer software), power source 18, and cables 19 operably connecting these elements effective to transfer informational signals and/or power between components. Further elements of a system 20 may include Doppler power source 21, audio monitor 23, video monitor 25 (which may be a computer monitor operably connected to a standard computer), analysis module 27 (which may be, or include, a standard computer and computer software), power source 29, RF power source 30, RF controller 31, ground pad 32 (for monopolar RF systems), and cables 33 operably connecting these elements effective to transfer informational signals and/or power between components.

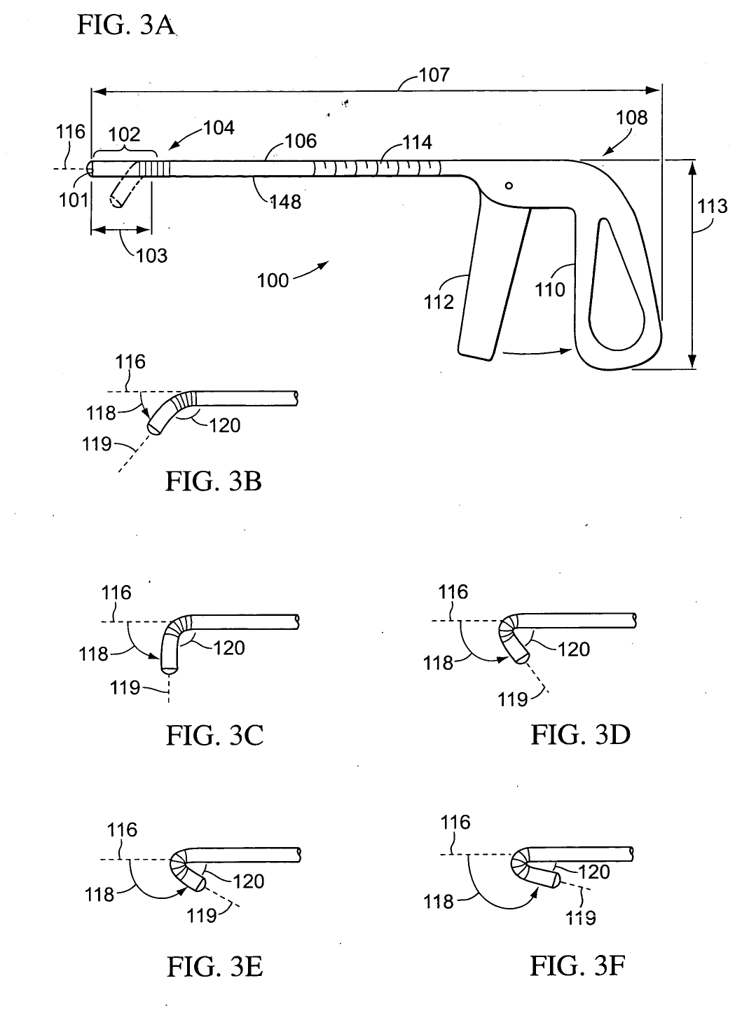

Arthroscopic probes having features of the invention are depicted as probes 12 and 22 in FIGS. 1 and 2 respectively, and as probe 100 in FIG. 3. As shown in the FIG. 3 (and analogously in FIGS. 1 and 2) such probes include a tip 101 and tip portion 102, having a tip portion length 103. The tip portion 102 includes a flexible region 104, and is disposed at the distal part of shaft 106. Probe 100 has a probe length 107. A handle 108 is disposed at the proximal end of the shaft 106, and includes a fixed handle portion 110 which may serve as a grip, and a movable handle portion 112 which may serve as, or connect with, an actuator. Handle 108 is configured to be readily held by a human hand, enabling an operator to manipulate a probe 102 and to manipulate a probe tip 102 by operation of the movable handle portion 112 effective to move the tip 102 with respect to shaft 106 as flexible region 104 flexes under operator control. Handle 108 has a handle length 113.

Elongated shaft 106 has a shaft axis 116 along the longitudinal direction of the shaft 106 as illustrated in FIG. 3A. Shaft 106 has an outer surface provided by shaft wall 148, and typically has a substantially cylindrical, elongated form, with a substantially circular cross section. A shaft 106 may be hollow, for example may be a hollow tube with a circular or oval cross-sectional shape. However, a shaft 106 of an arthroscopic probe 100 having features of the invention may have any suitable cross-sectional shape, including elliptical, triangular, square, rectangular, irregular, or may have another elongated configuration with any other cross-sectional shape.

As shown in FIGS. 1-3, an arthroscopic probe having features of the invention may have a mark 114 or markings 114 along a portion of a shaft 106, or along an entire shaft 106, and/or provided on a tip portion 102 or on the tip 101. Markings 114 are useful to aid an operator in determining the position or depth of the device or degree of deflection of tip. A mark 114 is typically visible by eye by an operator or observer, although in embodiments a mark 114 may also be radiopaque or otherwise detectable by means other than the human eye. Such markings may comprise colored portions of the shaft. Such markings may be all of a single color or may include multiple colors, and may be lines, bands, dots, geometric shapes, symbols, colors, combinations of these, or other indications detectable by an operator during use of an arthroscopic probe 100 having features of the invention. Markings 114 may completely circle the shaft or may only occupy a part of a shaft circumference. Such markings are useful to aid an operator in judging the depth or position of the tip portion of the probe during use. Markings 114 may be useful, for example, to indicate the position of the tip 101 and tip portion 102 with respect to the skin surface of a patient into which the instrument has been inserted. In embodiments, where the shaft 106 is transparent or has a transparent portion allowing an operator to see a control rod 156 within the shaft, markings 114 may also be carried on the control rod 156 to aid an operator in determining the degree of tip deflection during operation of the device.

Arthroscopic devices having features of the invention include a movable tip capable of flexing or bending. Flexing or bending of the tip is effective to place the tip at a position away from a position directly along a longitudinal axis of the shaft. As illustrated in FIGS. 3B-3F, flexion of flexible region 104 causes tip 101 and tip portion 102 to assume positions defining a deflection angle 118 with respect to shaft axis 116. The amount of deflection denoted by a deflection angle 118 is taken with respect to the longitudinal axis 116 of the probe 100 and measured by the number of degrees between the longitudinal axis 116 of the probe and a line taken along the longitudinal axis 119 of the tip portion 102. With tip 101 disposed along shaft axis 116, prior to flexion of flexible region 104, shaft axis 116 and tip portion axis 119 are parallel, and the deflection angle 118 is 0°.

The amount of angular flexion of the tip 101 from the longitudinal axis 116 may be, for example, as much as about 170°, or about 150°, or about 120°, or about 90°, or about 45°. The portion of the shaft 106 proximal to the distal tip 101 that exhibits the greatest degree of flexion is within the flexible region 104 of the shaft 106. FIGS. 3B-3F are schematic illustrations of flexion of a tip portion 102 of a hip probe 100 of FIG. 3A, in which the tip 101 is displaced from a position along shaft axis 116 by different amounts. FIGS. 3B-3F thus illustrate different examples of tip 101 movement, showing ranges of motion that may be provided by a hip probe 100 having features of the invention.

Increasing downward movement of tip 101 and tip portion 102 with respect to shaft 106 and shaft axis 116 increases deflection angle 118 (shown in the figures as counterclockwise rotation), as indicated in FIGS. 3B-3F, until deflection angle 118 approaches 180° as tip moves around to almost touch shaft 106 as shown in FIG. 3F. The complementary angle 120 is 180° with tip 102 disposed along shaft axis 116, prior to flexion of flexible region 104, and decreases as the tip rotates in the direction shown in the figures as counterclockwise.

FIGS. 3B-3F show tip portion displacements of about 45°, about 90°, about 120°, about 150°, and about 170°, respectively. Tip movement of deflection angles greater than about 90°, as illustrated in FIGS. 3D, 3E, and 3F, are great enough that the distal portion of the tip is directed backwardly towards the handle portion of the probe.

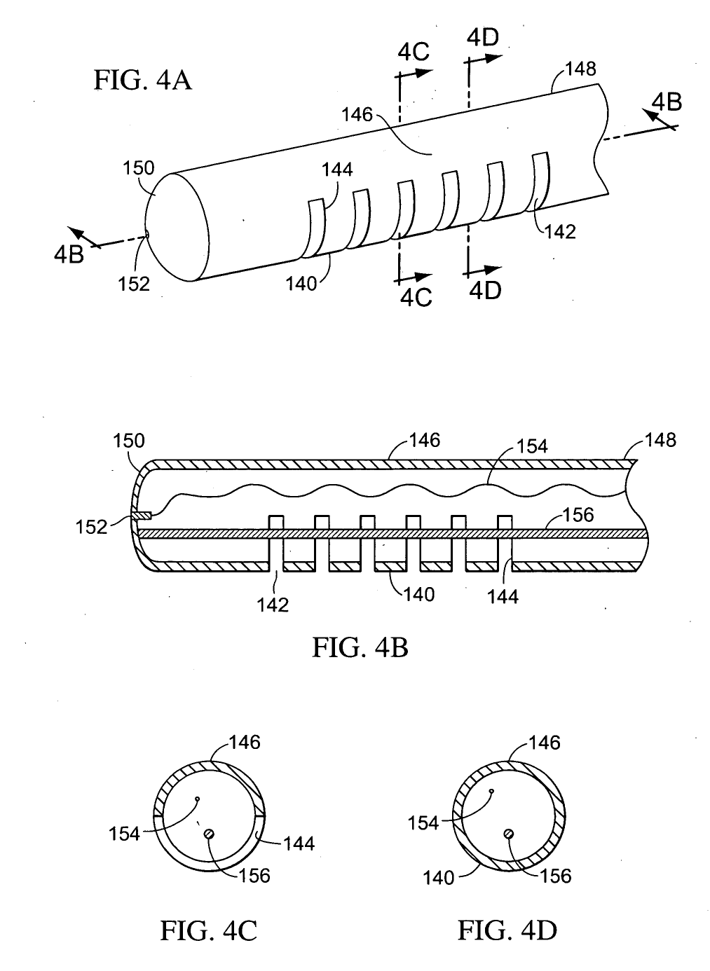

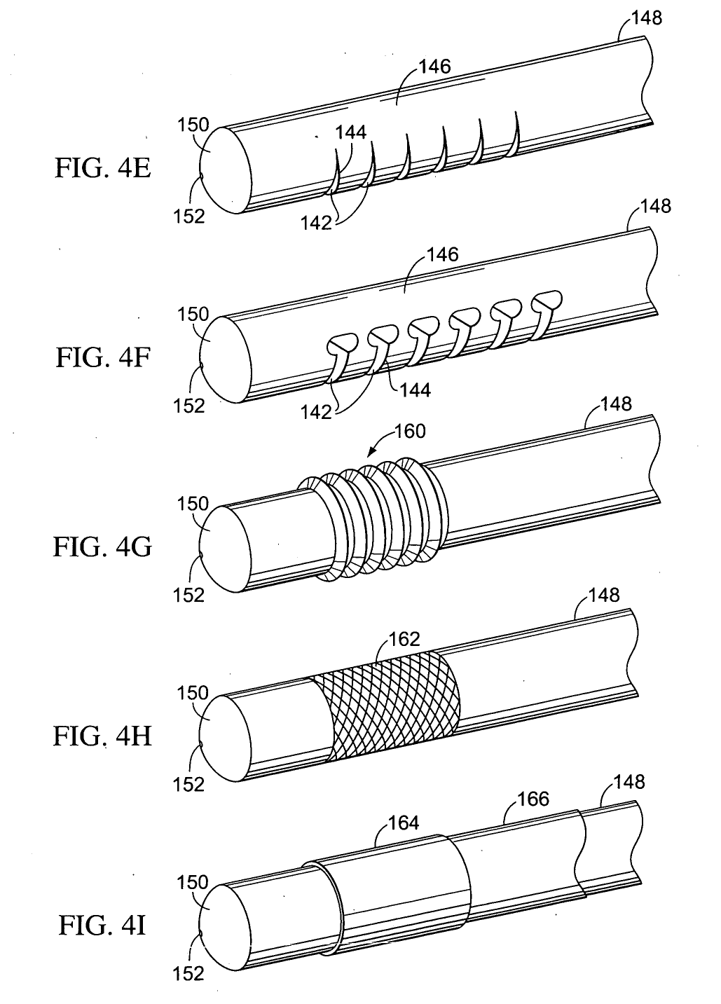

A flexible portion 104 of the shaft 106, part of the tip portion 102, allows for the movement of the tip 101. The flexible portion 104 may include gaps in portions of the shaft, a hinge, a pleated or accordion-type structure, a mesh, a flexible material, a combination of two or more of such elements or means for providing flexibility or movability, or other means and elements effective to allow or provide for movement of the tip. Examples of tip portions 102 including flexible regions 104 are shown in FIG. 4.

In embodiments, a flexible region 104 may include a substantially cylindrical portion in which, on one side of the portion, the substantially cylindrical walls are interrupted by gaps or are missing. For example, a substantially cylindrical wall may include a cut-out portion, or several cut-out portions, as illustrated in FIG. 4A. FIG. 4A shows a distal tip portion 102 with a flexible region 104 having ridges 140 separated by circumferential gaps 142. Ridges 140 have ridge walls 144 defining the gaps 142 in which partial diameters of the flexible region 104 are absent, so that, in a longitudinal direction along the walls of the flexible region 104, the wall material is discontinuous. There remains, however, even within the flexible region 104 of the tip portion 102, a continuous portion 146 of the shaft wall 148. For example, the wall material may be discontinuous around more than about 90°, or more than about 180°, or more than about 270° of angle around the circumference of the cylindrical wall 148. FIG. 4B is a cut-away view of the flexible region 104 shown in FIG. 4A, in a longitudinal view. FIGS. 4C and 4D are each views showing a cross-section of a flexible region 104 of a tip portion 102 as viewed from a distal position looking proximally, illustrating the absence of material within a gap 142 (4C, lower portion of circumference, showing ridge 140 proximal to the gap 142) and the continuous material making up a ridge 140 (4D).

Also shown in FIG. 4B are other features of a hip probe 100 having features of the invention, such as a Doppler element 152 disposed on the tip 150. A Doppler element 152 may be a Doppler radiation source configured to emit radiation suitable for Doppler detection of movement such as blood flow in a blood vessel, a Doppler sensor configured for Doppler detection of movement such as blood flow in a blood vessel, or both, such as a Doppler transducer. A Doppler element 152 is preferably operably connected to other elements, such as, for example, a power source, a signal source, a signal analyzer, an audio and/or video monitor, or other element, via a cable(s) or wire(s) 154.

Movement of a tip 101 and a tip portion 102, including flexion of a flexible region 104, may be initiated and controlled by, for example, a control element 156 as illustrated in FIG. 4B. A control element 156 is an elongated element configured to be able to pull on tip portion 102 when tip flexion is desired, and may be a rod, cable, tube, or other element that is more flexible in a transverse direction than in a longitudinal direction. A control element 156 may be attached to a tip portion 102 by any suitable means, including welding, gluing, by means of threads, notches or hooks, and may, for example, attach to an inner surface of a tip 101, as illustrated in FIG. 4B, may attach to a distal gap 142 (e.g., by a bent or hook portion on the end of control element 156 engaging with a gap 142) or may attach by any suitable method of attachment. Where the hip probe 100 is a Doppler-RF hip probe 100, tip portion 102 also includes at least one RF electrode 158, which is also operably connected to other elements via an electrical wire or wires 154.

Gaps 142 may be delimited by ridge walls 144 that are angled with respect to a shaft circumference, as illustrated in FIG. 4E. The angle subtended by the gaps 142 may vary from small angles to large angles, and serves to determine the direction, extent, and ease with which the tip portion 102 will flex upon urging by a control rod 156, such as a cable, rod, pulley, gear, or other mechanical means for applying force to a tip portion 102.

Gaps 142 may separate ridges 140 having ridge walls 144 that are have curved as well as straight edges, and may be gaps 142 with widths that differ at different parts of the gap (see, e.g., FIG. 4E). Gaps 142 may separate ridges 140 with walls 144 that have wider portions and thinner portions. For example, a gap 142 may have wider portions at the ends of the gap 142, for example, than at the mmid-ppoint of the gap, as illustrated in FIG. 4F. A gap 142 may extend for only a small portion of a circumference (a “shallow” gap), or may extend for a large fraction of a circumference (termed a “deeper” gap). Such deeper gaps or wider portions may increase the ease or range of motion of a tip portion 102 as compared to a tip portion 102 lacking gaps 142 with such deeper gaps or wider portions. A gap 142 having a wider portion may be defined by ridges 140 which are substantially parallel to a circumference of the shaft 106, or which are angled with respect to a diameter of the shaft 106, or combinations of these. A flexible portion 104 having multiple gaps may have gaps of different depth, width, angle, shape, or configuration.

A flexible portion 104 may have pleated walls, with pleats 160, where the circumference of the walls varies longitudinally along the tip portion, for example, as illustrated in FIG. 4G. Such pleats are able to flex and extend longitudinally, or to flex and contract (e.g., fold up) longitudinally, as may be required by a deflection or angling of the tip portion 102. As a pleated flexible region 104 bends, the outward portion of the flexible region 104 (defined as the region that is stretched as the deflection angle increases) extends longitudinally and the inner portion (defined as the region that is compressed as the deflection angle increases) contracts longitudinally. Such flexion may be caused by, for example, urging by an interior cable, rod, pulley, gear, or other mechanical means.

A further example of a movable tip portion 102 is shown in FIG. 4H, in which a tip portion has a wall 148 having mesh 162 in portions of the shaft wall 148. Such a mesh 162 may be a metal mesh, such as a braided wire (e.g., stainless steel) or may be a hydrocarbon polymer, a fluorinated hydrocarbon, a silicone rubber material, or a glass mesh material. An alternative configuration suitable for flexing a tip portion 102 uses flexible material to allow for tip movement, as shown, in FIG. 4I. A portion of the shaft wall of the movable tip of FIG. 4I includes a flexible material. Such a flexible material may be, for example, a fluorinated hydrocarbon such as polytetrafluoroethylene (PTFE, also known as TEFLON®), or an elastomeric material such as silicone rubber, or a polymeric hydrocarbon material such as, for example, polyolefin, polyurethane or polyethylene, or other suitable material. A tip portion 102 may include more than one of the materials or configurations discussed herein and shown in these figures. For example, a tip portion 102 having gaps 142 or pleats 160 in portions of the tip wall 148 may be made with a flexible material.

The shape, depth (portion of the shaft circumference removed), width, and spacing of the gaps affect the amount of deflection of the flexible region. For example, wider gaps 142 at the flexible region 104 of the shaft 106 allow greater amounts of deflection than would otherwise be possible. Smaller amounts of material between gaps 142 (thinner ridges 140) also allows greater amounts of deflection. Gaps 142 may separate ridges 140 having parallel sides (ridge walls 144 aligned substantially along a circumference of the shaft, e.g., as illustrated in FIGS. 4A-4D), or may separate ridges 140 having ridge walls 144 that are aligned at an angle with respect to a circumference (e.g., as illustrated in FIG. 4E). Alternatively, or in addition, a flexible region 104 may comprise a portion having pleats 160 (FIG. 4F), a portion comprising a mesh 162 as illustrated in FIG. 4G, a portion comprising a flexible material 164 (e.g., as illustrated in FIG. 4H), or a combination of two or more of these means for allowing an angled portion to deflect. Gaps 142, pleats 160, mesh 162, flexible materials 164, combinations of these, or other means that increase the ability of an flexible region 104 to flex and to deflect or move a tip portion 102 also allow deflection of an flexible region 104 with lesser amounts of force than would otherwise be required.

Movement of the tip portion 102 of the device is controlled by a control element, such as a handle 108, piston 174, plunger 176, or other element suitable for use by an operator, such as a surgeon, during an arthroscopic procedure. A handle 108, piston 174, plunger 176 or other element is preferrably configured to be readily operable by hand or is connected to an element configured for use by hand. A handle 108 may be, for example, an element of a lever mechanism operably connected to the tip of the device by means of cables, rods, tubes, wires or other elements or means disposed within or along the shaft, effective that movement of the handle portion may initiate and control the extent of movement of the tip portion. For example, a control element 156, shown as a flexible rod, is shown in FIG. 4B. A control element 156 may connect to, and impel or otherwise direct the movement of, a piston 174, a plunger 176, in order to direct and control the movement of a tip portion 102. In embodiments, a control element 156 may connect directly to a tip portion 102, for example, to an interior portion of a tip portion 102, and directly impel or otherwise direct the movement of the tip portion 102. A piston 174 or a plunger 176 may be, for example, an element of a hydraulic or electronic mechanism operably connected to the tip of the device by means of tubes, cables, rods, wires or other elements or means disposed within or along the shaft, effective that movement of the piston 174 or plunger 176 initiates and/or controls the extent of movement of the tip portion.

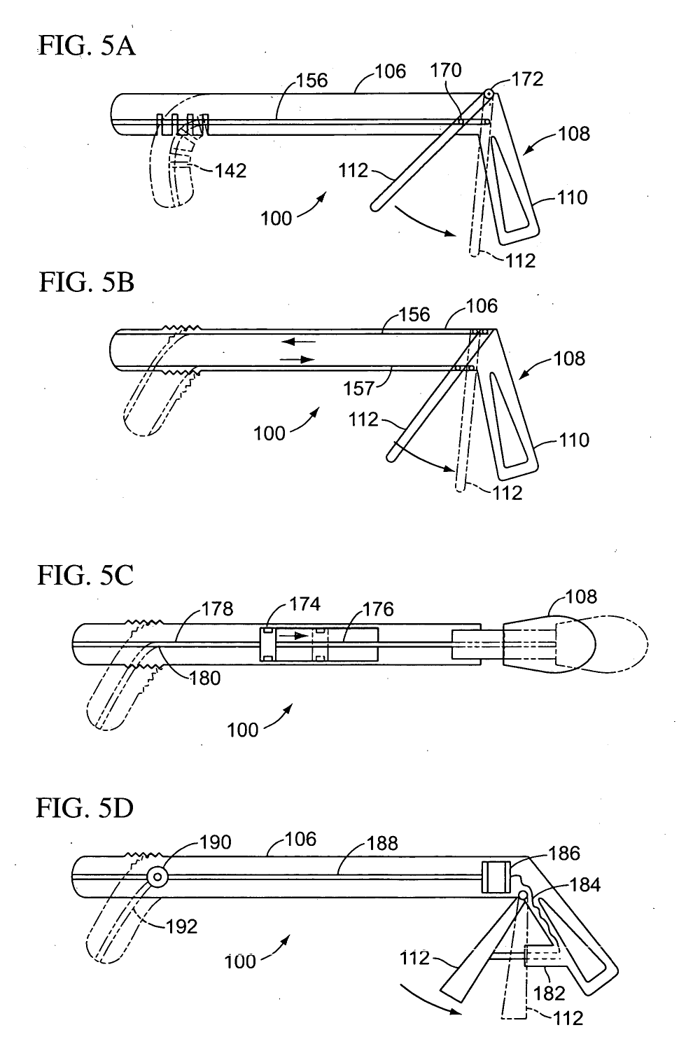

Examples of mechanisms for moving the tip portion of a Doppler hip probe having features of the invention are illustrated in FIGS. 5A-5D. A Doppler hip probe 100 having features such as those illustrated in any of FIG. 5A, 5B, 5C, or 5D may be either a Doppler hip probe 100 or a Doppler-RF hip probe 100. The initial, or resting configurations of the examples of Doppler hip probes 100 and Doppler-RF hip probes 100 shown in these figures are indicated by solid lines. Deflection or other motion of the tip portion 102 is shown in these figures by dotted lines indicating the tip configuration after movement of the handle portion 108. Thus, an operator may control the configuration of these devices by manipulation of the handle portion 108 of these devices 100.

FIG. 5A shows a hip probe 100 in which the movable tip 102 is directed to deflect in a downward direction by a mechanical mechanism controlled by motion of a handle portion 108. Handle portion 108 includes a fixed handle portion 110 and a movable handle portion 112. A fixed handle portion 110 may serve as a grip, or base to aid manual control, orientation, manipulation and operation of a hip probe 100 by an operator. Movable handle portion 112 is configured for manual operation by the operator of a hip probe 100, and is operably connected to tip portion 102 effective to control the position and placement of tip portion 102. Thus, a movable handle portion 112 may serve as an actuator or control for initiating and controlling the movement of tip portion 102 with respect to shaft 106 of a hip probe 100 having features of the invention. Tip portion 102 may be operably connected to a handle portion 108 having a movable handle portion 112 by mechanical means, including hydraulic, pneumatic, or other mechanical means, electrical means, or other operable connection.

Such mechanical means may include a mechanical mechanism such as a lever mechanism (here shown attached to the handle) which may push or pull a rod, tappet, or other element effective to deflect a tip portion 102 in a downward direction upon movement of the handle 110. Such a rod, tube, cable, or other element, such as a control element 156, may be attached to a tip portion 102 by any suitable means, including welding, gluing, by means of threads, notches or hooks, and may, for example, attach to an inner surface of a tip 101, may attach to a distal gap 142, or may attach by any suitable method of attachment. Greater amounts deflection of the tip portion 102 and of the bending of the flexible region 104 of the shaft 106 increase the deflection angle 118 (and decrease the complementary angle 120) transverse to the shaft axis 116 defining a longitudinal direction.

As illustrated in FIG. 5A, a hip probe 100 (shown in longitudinal cross-section in this figure) has a control rod 156 within a hollow shaft 106 for controlling the tip portion 102. Control rod 156 may be attached to a portion of a movable handle portion 112 at an attachment point 170 operably connecting control rod 156 with movable handle portion 112 effective that movement of movable handle portion 112 moves control rod 156 and so controls the position and orientation of tip portion 102. A movable handle portion 112 may be movably connected with a pivot point 172, as shown in FIG. 5A, effective that proximal movement of the movable handle portion 112 imparts proximal movement to control rod 156 attached to handle portion 112 at attachment point 170. The load configuration illustrated in FIG. 5A is that of a second class lever. In embodiments, a pivot point 172 may be in other positions with respect to control rod 156 and movable handle portion 112, so that first class, second class, or third class levers are provided in a handle mechanism comprising an attachment point 170, a pivot point 172, and handle portions 110 and 112.

As discussed above, and as illustrated in FIGS. 4A-4F, movement of the tip 101 may be aided by, and is also directed in part by gaps 142 in the wall 148 of the tip portion 102. Such gaps 142 are similar to those illustrated in FIGS. 4A-4F.

Thus, for example, a hip probe 100 having features of the invention, and configured as illustrated in FIG. 5A, may have a hollow shaft 106 enclosing a flexible control rod 156 which is connected to the handle mechanism 108, in particular, movable handle portion 112, at its proximal end, and connected near, or to, the distal tip 102 of the shaft 106 at its distal end. Since the control rod 156 is fixed at a distal position near or at the distal end of the shaft 106, proximal movement of the control rod 156 pulls tip 102 backwardly towards the handle 108. Gaps 142 in the wall 148 of the shaft 106 make the downwardly facing portion of the shaft 106 weaker and more flexible than the opposite, upwardly facing portion of the shaft 106 (that includes continuous portion 146 of wall 148). Proximal movement of the control element 156 pulls the tip portion 102 of the shaft 106 proximally, causing the flexible region 104 of the shaft 106 to increase the amount of its bend (increase deflection angle 118 and decrease complementary angle 120). Greater amounts of pressure on the handle increase the extent of proximal movement of the flexible control element 156, and increase the extent of the deflection of the tip portion 102 and of the bending of the flexible region of the shaft (increase the deflection angle 118). A flexible control element 156 as illustrated in FIG. 5A may be made of any suitable material, such as stainless steel or other metal, polymer, fiberglass, composite, or other material having suitable rigidity on a longitudinal direction and suitable flexibility transverse to the shaft axis 116 defining a longitudinal direction.

A Doppler element 152, and optionally a RF electrode 158 or RF electrodes 158, is disposed on the distal tip 101 of the shaft 106. In embodiments, a Doppler element 152, and/or one or more RF electrodes 158 may be disposed in a tip portion 102 at a position proximal to tip 101. The shaft may also enclose wires 154 or other connecting elements attached to the Doppler element 152 and/or RF electrode(s) 158 in addition to the control rod 156. Alternatively, such wires 154 may be disposed along an outer surface of the shaft 106, or between the shaft wall 148 and a shaft wall coating 166 (such as a sleeve or a coating, which may be an insulating sleeve or coating, which may be painted on, taped, rolled on, shrunk-fit, or otherwise placed around and onto the shaft 106). A sleeve or coating may be made of, or include, any suitable material, such as a polyolefin material.

As illustrated in FIG. 5B, as in 5A, a mechanical mechanism may include a lever mechanism (here shown attached to the handle 108) which may push or pull control rod 156 (which may be a rod, tappet, cable, or other element) effective to deflect a tip portion 102 in a downward direction upon manipulation of the handle 108. However, in 5B there is also a mechanical mechanism including a lever attached to the handle 108 which may push a first control element 156 (which may be a cable, rod, tappet, or other element) forwardly and pull a second control element 157 (which may be a cable, rod, tappet, or other element) backwardly effective to deflect a tip portion 102 in a downward direction upon movement of the handle 108. Both such mechanisms operate together as shown in FIG. 5B, or each may operate alone and independently of the other as illustrated in FIG. 5A.

In embodiments of a hip probe 100 as illustrated in FIG. 5B, the first and second control elements 156 and 157 may be connected at one or both ends to form a continuous element such as a loop of cable that runs along the shaft of the probe. In embodiments, such a loop of cable is enclosed within the shaft 106, and both ends are attached to the probe tip 101 or tip portion 102. In embodiments, such a loop passes over a gear or through a pulley element at or near the flexible region of the shaft, providing mechanical advantage effective to amplify the amount of deflection at the flexible region. It will be understood that other mechanical elements and mechanisms, with or without levers, may be linked to a handle or other actuating mechanism effective to urge or control the movement of a tip portion 102 of a Doppler hip probe 100 having features of the invention.

Further examples of Doppler hip probes 100 having features of the invention are shown in FIGS. 5C and 5D. As shown in FIGS. 5A, 5B and 5D, a handle portion 108 may include a trigger or lever mechanism 112, or as shown in FIG. 5C, a handle portion 108 may be configured similar to a syringe and include a piston 174 or plunger 176. In any case, a handle 108 may include a fixed portion 110 and a movable portion 112 configured for operation by a hand, or a portion of a hand, such as a finger, thumb or fingers, of an operator.

FIG. 5C shows an embodiment of a hip probe 100 having features of the invention in which the movable tip 101 is directed to deflect in a downward direction by a mechanical mechanism controlled by motion of a syringe-type handle portion 108, proximal movement of which moves piston 176 and plunger 174 so as to remove fluid (liquid or gas) present within chamber 178 so as to reduce pressure within flexible element 180 which curves downwardly upon release of tension that had been caused by the pressure of the fluid, effective to produce a downward deflection of the tip portion 102. Such a flexible element 180 is configured so that its least-stressed configuration is curved, as indicated by dotted lines in FIG. 5C, and is able to assume a straight configuration (indicated by solid lines in FIG. 5C) with pressure applied internally. Chamber 178 is configured to contain and allow flow of fluid into and out of flexible element 180. A flexible element 180 may be made with any suitable resilient material, such as a metal such as spring steel, or may be a resilient plastic, polymer, composite, or other material.

In mechanisms such as the one illustrated in FIG. 5C, the flexible element 180 is curved in the absence of internal pressure, and is maintained in a straight configuration (e.g., substantially along a longitudinal axis 116 of the shaft 106) by pressure, such as hydraulic or pneumatic pressure. Such pressure may be provided, for example, by distal movement of the piston 174 attached to the plunger 176 and handle 108 shown in the figure, and may be reduced or removed by proximal movement of the piston 174, as indicated in the figure.

FIG. 5D shows a partially cut-away view of a hip probe 100 configured for use in arthroscopic evaluations and treatments of the hip and having features of the invention, in which the movable tip 101 is directed to deflect in a downward direction by an electrical mechanism controlled by manipulation of handle portion 108. Proximal movement of movable handle portion 112 (controlled by the operator) operates a switch 182 producing a signal sent along a wire 184, shown within the fixed handle portion 110 in FIG. 5D, which activates motor 186 rotating threaded shaft 188 effective to rotate gear 190 attached to control rod 192 within tip portion 102 effective to deflect the tip portion 102. In embodiments, the length of time that an operator manipulates handle portion 112, causing rotation of threaded shaft 188, provides control for the amount of deflection of tip portion 102. In embodiments, switch 182 is configured to provide a control signal, and motor 182 is a stepper motor, so as to displace the tip portion 102 by an amount proportional to the control signal produced by the switch 182 under the control of the operator. The examples shown in FIGS. 5A-5D, and discussed herein, are suitable means and mechanisms for controlling the position of a tip portion 102 of a hip probe 100 having features of the invention. However, they are not the only mechanisms and mechanical means that are suitable for the practice of the invention. It will be understood that other mechanisms and mechanical means can also be used to control the amount of deflection of a tip portion 102 under control of an operator.

A shaft 106 of a hip probe 100 may be made of any suitable material, including metal, polymer, plastic, fiberglass, or other material or mixture of materials. A tip portion 102, which includes a flexible portion 104, may be made of any suitable material, including metal, polymer, plastic, fiberglass, or other material or mixture of materials, which have suitable flexibility. Such flexibility may be provided by providing appropriate wall thickness, by including, for example, gaps in the flexible portion 104, or by other modifications to the structure or material. It will be understood that, for a given material, smaller diameter and thinner walls typically provide greater flexibility than do thicker walls. Suitable metals for a shaft 106 and a tip portion 102 include stainless steel and nickel-titanium alloys, and suitable polymeric materials include polycarbonate, polyethylene, polyurethane, polyolefin, and other materials which may be used in fabrication of part or all of a shaft 106 or tip portion 102.

The tip portion 102 of an arthroscopic hip probe 100 having features of the invention includes a Doppler element 152 capable of receiving (and optionally capable of sending, e.g., emitting) radiation effective for use in the Doppler detection of blood flow, and is made of materials compatible for passing and receiving radiation used by a Doppler transducer. Such suitable materials are preferably of medical grade, and may be, for example, metals, metal alloys, plastic, polymer, polymer composite, or combinations of some or all of these materials. Metals may be, for example, stainless steel, silver, copper, gold, platinum, titanium, alloys of the preceding metals, or other metal or metal alloy. Preferable metal materials are good conductors of electricity. Plastic materials may include, for example, fluorinated hydrocarbons such as polytetrafluoroethylene (PTFE, also known as TEFLON®), vinyl, acrylic, polycarbonate, polyethylene, polyurethane, polyolefin, epoxy resin, nylon, silicone rubber, or other plastic. Plastic materials are preferably good electrical insulators. Other suitable materials include glass, additional polymeric materials, and other materials. Suitable materials are preferably sterilizable.

Where the Doppler energy is sonic or acoustic energy, such as ultrasound energy, the tip portion 102 may be made of or include, for example, metal, such as, for example, stainless steel, silver, copper, gold, platinum, titanium, alloys of the preceding metals, or other metal or metal alloy. Where the Doppler energy is electromagnetic energy, such as infrared energy, the tip portion 102 may be made of or include, for example, transparent materials such as transparent polymers or plastics, glass, acrylic, epoxy or other material or combination of materials transparent to the electromagnetic energy used for the Doppler detection of blood flow. In an embodiment, the tip portion 102 may include polycarbonate. Where the device includes both Doppler element 152 and RF electrodes 158 disposed on a tip portion 102, or at or near the tip 101, the tip portion 102 may include a metal portion for the RF elements such as RF electrodes 158 and a polymer, plastic, or glass portion associated with the Doppler element(s) 152 such as a Doppler transducer/receiver. For example, in embodiments with an RF electrode 158 in addition to a Doppler element 152, the tip portion 102 may be made with a metal alloy and have a polymer cap portion through which energy may pass for the Doppler measurements.

The tip portion 102 may have a flat end (e.g., substantially perpendicular to the longitudinal axis of the tip portion), or may have a rounded (e.g., dome-shaped) end, may have a faceted end having multiple flat faces disposed at angles to each other, or may have another shaped end. The tip portion 102 may have a complex shape, such as, for example, having a flat portion and a rounded portion. In embodiments, the tip portion 102 may have a flat portion configured for use as an RF electrode 158, and a dome-shaped portion configured for use with a Doppler transducer/receiver element 152. For example, a device may have a tip 101 having a flat portion configured for use as an RF electrode 158 surrounding a centrally-disposed dome-shaped portion configured for use with a Doppler transducer/receiver 152.

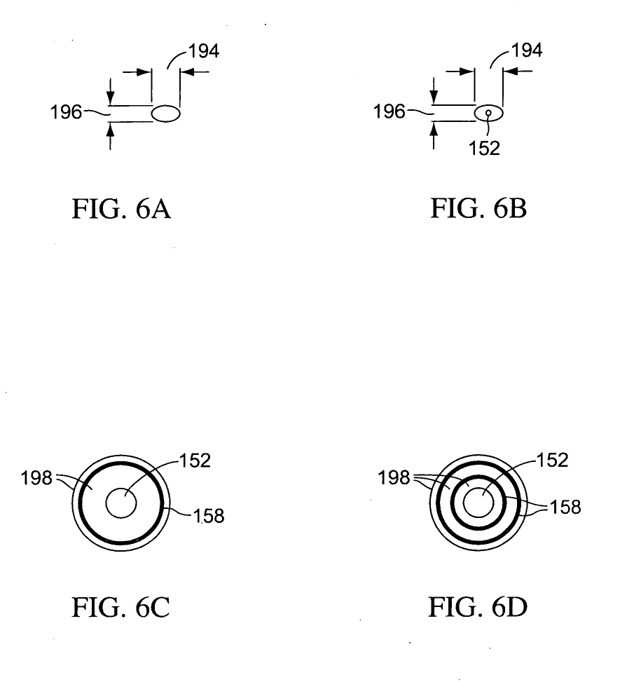

End-on views of examples of tips of arthroscopic probes having features of the invention are shown in FIG. 6. FIGS. 6A-6D show the distal tip 101 of a tip portion 102 of a Doppler probe 100 in schematic view as if viewed from distal point looking in a proximal (towards the handle) direction. FIG. 6A shows an example of a Doppler probe tip 101, while FIG. 6B shows a tip 101 of a Doppler-RF probe (monopolar RF) 101 having features of the invention. The Doppler probe examples of FIGS. 6A and 6B have tips 101 with elliptical cross-sections. As ellipses have major and minor axes, tips 101 shown in FIGS. 6A and 6B having elliptical cross-sections have major cross-sectional widths 194 and minor cross-sectional widths 196. In embodiments of a hip probe 100 having features of the invention, major cross-sectional widths 194 of a tip 101 may be from about 1 mm to about 20 mm, and in embodiments may be from about 3 mm to about 10 mm, and in further embodiments may be from about 4 mm to about 8 mm. In embodiments, minor cross-sectional widths 196 of a tip 101 may be from about 0.5 mm to about 15 mm, in further embodiments may be from about 1 mm to about 8 mm, and in yet further embodiments may be from about 2 mm to about 6 mm. In a particular embodiment of, a hip probe 100 having features of the invention, the major cross-sectional width 194 of a tip 101 may be about 5 mm and the minor cross-sectional width 196 of a tip 101 may be about 2 mm.

As discussed above, a shaft 106, a tip portion 102 and a tip 101 may have a circular cross-section. For example, tip portions having circular cross-sections may have cross-sectional diameters, as discussed above, of between about 2 mm and 20 mm. FIG. 6C shows a tip 101 of a Doppler-RF probe (monopolar RF) 100 having a circular cross-sectional shape, a Doppler transducer 152 disposed in a central position of the probe tip 101, and a layer of insulation 198 surrounding at least a portion of the RF electrode 158. FIG. 6D shows a tip 101 of a Doppler-RF probe (bipolar RF) 100 having a circular cross-section showing the Doppler transducer 152 disposed in a central position of the probe tip 101 and showing the two RF electrodes 158 of the bipolar RF probe 100 disposed around the Doppler transducer 152. Insulation 198 separates the bipolar RF probes 158 from each other and from the Doppler transducer 152.

A Doppler transducer 152 disposed on a probe tip 101 is configured for use in Doppler measurements and may be configured to emit energy suitable for use in Doppler measurements. A Doppler transducer 152 disposed on a probe tip 101 may be configured to receive energy for making Doppler measurements. Thus, in embodiments, a Doppler transducer 152 disposed on a probe tip 101 may be configured to emit and to receive energy, and is configured for use in Doppler measurements. Such energy may be acoustic energy (e.g., ultrasound energy of about 20 megaHertz (MHz)) or electromagnetic energy (e.g., infrared energy having a wavelength greater than about 1 micrometer (μm)).

A Doppler transducer 152 disposed on the tip portion 102 of a device 100 may have dimensions of about 1 mm to about 20 mm, or may have dimensions of from about 2 mm to about 10 mm. In embodiments, a Doppler transducer 152 disposed on a movable tip portion 102 of a device 100 having features of the invention may be between about 2 mm and about 4 mm in diameter.

Doppler transducers may include an ultrasound transducer comprising a crystal, typically a crystal having piezoelectric properties. Such a crystal may be attached (e.g., soldered) to a conductive wire or conductive wires effective to provide an operative electronic connection between the wires and the crystal, and thereby between the crystal and any electronic equipment attached to the wire or wires. As discussed in U.S. Pat. No. 6,974,416 (hereby incorporated by reference in its entirety), the orientation of a crystal ultrasound transducer may affect or control the direction in which it may operate. For example, where the orientation of the crystal is perpendicular to the longitudinal axis of the probe, the crystal detects motion substantially in one direction only. Where a Doppler ultrasonic crystal is oriented parallel with the longitudinal axis of the probe, the Doppler ultrasonic crystal is able to simultaneously detect flow from either direction, relative the probe tip. In embodiments of probes having features of the invention, the Doppler ultrasonic crystal is oriented perpendicular to the longitudinal axis of the probe. In embodiments of probes having features of the invention, the Doppler ultrasonic crystal is oriented parallel with the longitudinal axis of the probe.

In embodiments, a Doppler ultrasonic crystal is covered with or encased in a protective material, such as epoxy, to form the probe tip. Such a probe tip may be used to detect possible blood flow when near to or when applied directly to a blood vessel surface. The conductive wires connected to the Doppler ultrasound crystal extend proximally through the shaft. These wires may be covered with an insulator, and may, for example, be covered with a polymer sleeve. In embodiments, the wires terminate on and connect with a connector in the handle portion. In other embodiments, the wires exit the probe and continue either to a connector, or continue to connect with a Doppler signal generating unit. Where the wires terminate in a connector, the connector is configured to accept further connections which can provide electronic continuity with a Doppler signal generating unit. A Doppler signal generating unit, such as the COOK® Vascular Blood Flow Monitor, is suitable for generating a Doppler signal. For example, the COOK® Vascular Blood Flow Monitor generates a 20 MHz Doppler signal suitable for use with the probe to sense pulsative blood flow within a blood vessel within a distance of about 8-10 mm from the probe tip. Such movement may be detected by the Doppler ultrasound crystal, and the detection signal may be processed and converted to an audible signal by an analysis unit or other suitable device or algorithm. Such a detector is effective to measure relative flow velocity and to detect blood flow in a vessel, thereby allowing the identification of blood vessels. Where no blood flow is detected in an anatomical structure, the operator can deduce that either the structure is not a blood vessel, or is a blood vessel in which blood flow is occluded. Where a second Doppler ultrasound crystal is provided in the probe tip, along with wires and other suitable connections and controls, it is possible to measure actual flow rates within the vessel.

When present, an RF electrode 158 may be a low-power RF electrode, or may be a higher power RF electrode, and may be a high power RF electrode. A low power RF electrode may be configured for use with RF power levels of between about 0 watts (W) and about 75 W, or up to about 40 W. An RF electrode may be a monopolar RF electrode, such as a monopolar RF electrode configured for use with RF energies up to about 40 W, or may be a bipolar RF electrode configured for use with RF energies up to about 40 W. Where the RF electrode disposed on tip portion 102 is a monopolar RF electrode, the RF electrode is preferably used with a grounding pad in contact with the patient and disposed at a distance from the site of the arthroscopic procedure. Where the RF electrode disposed on a tip portion 102 is a bipolar RF electrode, the active RF electrode is preferably used with a ground electrode disposed within about 20 cm of the probe tip during an arthroscopic procedure, and in embodiments a ground electrode is disposed quite close to the active RF electrode (e.g., within a cm, or less, of the active RF electrode). For example, as illustrated in FIG. 6D, a ground electrode may be disposed within a few mm of the active RF electrode.

A typical range of RF energy suitable for use in medical or surgical procedures using a device having features of the invention may be, for example, about 40 W effective to raise tissue temperatures at or near the probe tip to about 75° C.

Thinness of an arthroscopic instrumentation provides ease of access to the target site, particularly where an access cannula is used during an arthroscopic porcedure. However, thinness contributes to fragility and possibly decreased sensitivity of the Doppler transducer element. The devices, systems and methods for arthroscopic hip procedures disclosed herein are longer than many other arthroscopic devices in order to provide complete arthroscopic access about the hip. The thickness of the devices and systems disclosed herein are configured to provide a reasonable compromise between durability and ease of use. For example, a shaft of a device having features of the invention may have a cross-sectional dimension of between about 2 mm and about 20 mm, as discussed above, and is preferably about 5 mm.

Systems for use with arthroscopic hip procedures may, in addition the arthroscopic devices disclosed herein, include cables and connectors for delivering power for a Doppler transducer/receiver and/or for an RF electrode, or for delivering acoustic or electromagnetic energy from an energy source for use in Doppler measurements, or for delivering RF energy from an RF energy source to an RF electrode, a ground pad or ground electrode for use with an RF electrode, and/or other power or energy sources suitable for use with the devices and systems disclosed herein. Systems for use with arthroscopic hip procedures may also include an amplification system, a recording system, a power regulator, an audio monitor, a visual monitor, and other devices and elements useful with the devices and systems disclosed herein.

Devices having features of the invention are preferably disposable devices, and methods of using such devices may be methods in which each device is disposed of after use. Disposability is preferred for sterility purposes, and also provides the advantage of reducing the risk of breakage of a device during a procedure as new devices are used with each procedure. In embodiments, the device is a disposable, single use arthroscopic probe, capable of extended passage through the deeper soft tissues of the hip via a standard arthroscopic portal.

In embodiments, a method of performing an arthroscopic procedure at or near to a hip joint of a human patient, comprises

a) inserting a hip probe into a patient near a hip joint of the patient, said hip probe having a shaft and a Doppler probe disposed on a movable tip of said shaft;

b) moving said movable tip with respect to the axis of said shaft of said hip probe effective to place said Doppler probe adjacent a blood vessel of said patient near a hip joint of said patient; and

c) monitoring blood flow in said blood vessel.

In embodiments of the methods, the hip probe is a hip probe having features of the invention, such as, for example, a hip probe having a movable tin and a Doppler transducer, and may have an electrode for providing radiofrequency energy. The methods may further comprise a step d) applying radiofrequency energy to tissue near a hip joint of said patient.

Although cannulas are not required for surgical joint access, some surgeons may prefer to access the joint via cannulas. A probe having features of the invention may be used with an access cannula, or may be used without using an access cannula. A cannula optionally may be provided in a system having features of the invention, for use in situations where a cannula may be helpful, and by those surgeons who prefer to use a cannula in arthroscopic procedures. Thus, in embodiments of the methods of the invention, methods such as those discussed above may further include steps of providing a cannula, placing a cannula in position in a patient, and inserting a device having features of the invention into a cannula effective to position the tip of the device at a desired location within a patient.

Such methods including a cannula therefore may include, for example, the following steps:

a) placing a cannula in position in a patient;

b) inserting a hip probe into said cannula;

c) inserting said hip probe in said cannula into a patient near a hip joint of the patient, said hip probe having a shaft and a Doppler probe disposed on a movable tip of said shaft;

d) moving said movable tip with respect to the axis of said shaft of said hip probe effective to place said Doppler probe adjacent a blood vessel of said patient near a hip joint of said patient; and

e) monitoring blood flow in said blood vessel.

The methods including use of a cannula may further comprise a step f) applying radiofrequency energy to tissue near a hip joint of said patient.

Devices and systems embodying features of the invention may also include other useful features that may aid in identifying anatomic features and in distinguishing between vascular and neuronal structures within a patient.

While particular forms of the invention have been illustrated and described, it will be apparent that various modifications can be made without departing from the spirit and scope of the invention. Accordingly, it is not intended that the invention be limited, except as by the appended claims. Reference to the terms “members,” “elements,” “sections,” “portions,” and terms of similar import in the claims which follow shall not be interpreted to invoke the provisions of 35 U.S.C. §112, paragraph 6 unless reference is expressly made to the term “means” followed by an intended function.

1. An arthroscopic probe for a hip procedure having a shaft with a longitudinal axis defining a proximal direction, a distal direction and radial directions perpendicular to said longitudinal axis, a handle portion with a proximal end and a control element disposed at a proximal end of said shaft, said shaft having a tip portion with a distal tip and a Doppler transducer effective for detecting blood flow in a blood vessel, the distance between said distal tip and said handle proximal end defining a probe length, said control element being operably connected with said tip portion, the tip portion being movable with respect to said longitudinal axis at a deflection angle to the shaft, said deflection angle being controllable by the control element.

2. The arthroscopic probe of claim 1, wherein said deflection angle is between about 0° and about 170°.

3-4. (canceled)

5. The arthroscopic probe of claim 1, wherein said deflection angle is between about 0° and about 90°.

6. (canceled)

7. The arthroscopic probe of claim 1, wherein said handle is disposed at least partially along a radial direction, said handle radial direction defining a downward direction, and wherein said tip is configured to be able to move in said downward direction.

8-12. (canceled)

13. The arthroscopic probe of claim 1, wherein said tip portion has a flexible region proximal to said distal tip, and said shaft portion has a supporting shaft portion adjacent said flexible region of said tip portion, wherein said tip portion is movable when at least part of said flexible region moves with respect to said supporting shaft portion, the distance between the distal tip of said probe and the supporting shaft portion defining a tip length.

14. The arthroscopic probe of claim 13, wherein said tip length is between about 2 mm and about 75 mm.

15-16. (canceled)

17. The arthroscopic probe of claim 13, wherein said tip length is between about 15 mm and about 25 mm.

18. The arthroscopic probe of claim 1, wherein said tip portion has a substantially circular cross-sectional shape, said circular cross-sectional shape having a tip diameter.

19. The arthroscopic probe of claim 18, wherein said tip diameter is between about 2 mm and about 20 mm.

20. (canceled)

21. The arthroscopic probe of claim 18, wherein said tip diameter is between about 4 mm and about 8 mm.

22. The arthroscopic probe of claim 1, wherein said tip portion has a substantially elliptical cross-sectional shape, said elliptical cross-sectional shape having a larger elliptical diameter and a smaller elliptical diameter.

23. The arthroscopic probe of claim 22, wherein said larger elliptical diameter is between about 3 mm and about 10 mm and said smaller elliptical diameter is between about 1 mm and about 8 mm.

24. The arthroscopic probe of claim 22, wherein said larger elliptical diameter is between about 4 mm and about 8 mm and said smaller elliptical diameter is between about 2 mm and about 6 mm.

25. The arthroscopic probe of claim 1, wherein said probe length is between about 100 mm and about 500 mm.

26. (canceled)

27. The arthroscopic probe of claim 25, wherein said probe length is between about 250 mm and about 350 mm.

28. The arthroscopic probe of claim 1, wherein said Doppler transducer comprises an ultrasound source.

29. The arthroscopic probe of claim 1, wherein said Doppler transducer comprises an infrared radiation source.

30. The arthroscopic probe of claim 1, further comprising an electrode configured to provide radiofrequency energy to tissue.

31. The arthroscopic probe of claim 30, wherein said electrode configured to provide radiofrequency energy to tissue is selected from the group consisting of a monopolar radiofrequency electrode and a bipolar radiofrequency electrode.

32. (canceled)

33. The arthroscopic probe of claim 28, further comprising an electrode configured to provide radiofrequency energy to tissue.

34. The arthroscopic probe of claim 33, wherein said electrode configured to provide radiofrequency energy to tissue is selected from the group consisting of a monopolar radiofrequency electrode and a bipolar radiofrequency electrode.

35. (canceled)

36. The arthroscopic probe of claim 29, further comprising a source of radiofrequency energy.

37. The arthroscopic probe of claim 36, wherein said source of radiofrequency energy is selected from the group consisting of a monopolar source of radiofrequency energy and a bipolar source of radiofrequency energy.

38. (canceled)

39. An arthroscopic probe for a hip procedure having a shaft with a longitudinal axis defining a proximal direction, a distal direction and radial directions perpendicular to said longitudinal axis, a handle portion with a proximal end, said shaft having a tip portion with a distal tip and a Doppler means for detecting blood flow in a blood vessel, the distance between said distal tip and said handle proximal end defining a probe length, said control element being operably connected with said tip portion, the tip portion being movable with respect to said longitudinal axis at a deflection angle to the shaft, said deflection angle being controllable by a control means.

40. The arthroscopic probe for a hip procedure of claim 39, further comprising a radiofrequericy means for providing radio frequency energy for use during said hip procedure.

41. A system for a hip procedure, comprising an arthroscopic probe of claim 1, and another element selected from one or more of a power supply for a Doppler probe, an audio monitor output for a Doppler probe, a visual monitor output for a Doppler probe, a processing device for processing signals from a Doppler probe, and an access cannula.

42. A system for a hip procedure, comprising an arthroscopic probe of claim 30, and another element selected from one or more of a power supply for a Doppler probe, an audio monitor output for a Doppler probe, a visual monitor output for a Doppler probe, processing device for processing signals from a Doppler probe, a power supply for a radiofrequency probe, a grounding pad for a monopolar radiofrequency probe, a control for a radiofrequency probe, and an access cannula.

43. A method of performing an arthroscopic procedure at or near to a hip joint of a human patient, comprising:
inserting a hip probe into a patient near a hip joint of said patient, said hip probe having a shaft and a Doppler probe disposed on a movable tip of said shaft;
moving said movable tip with respect to the axis of said shaft of said hip probe effective to place said Doppler probe adjacent a blood vessel of said patient near a hip joint of said patient; and
monitoring blood flow in said blood vessel.

44. The method of claim 43, wherein said hip probe is a hip probe of claim 1.

45. The method of claim 43, wherein said hip probe is a hip probe of claim 28.

46. The method of claim 45, further comprising the step of applying radiofrequency energy to tissue near a hip joint of said patient.

47. The method of claim 46, wherein said tissue near a hip joint of said patient is selected from the group consisting of connective tissue, bone, muscle tissue, and vascular tissue.

48-50. (canceled)

51. The method of claim 47, wherein said tissue comprises vascular tissue selected from the group consisting of arterial tissue and venous tissue.

52. (canceled)

53. The device of claim 1, wherein said device is a disposable device configured for single use.

54. The device of claim 30, wherein said device is a disposable device configured for single use.

55. The device of claim 39, wherein said device is a disposable device configured for single use.

56. The device of claim 40, wherein said device is a disposable device configured for single use.

57-58. (canceled)
